(19) United States
(12) Patent Application Publication
(10) Pub. No.: US 2013/0107212 A1
(43) Pub. Date: May 2, 2013

(54) THREE-AXIS POSITIONING DEVICE AND METHOD FOR OPHTHALMIC EXAMINATION INSTRUMENT

(71) Applicants: Chun Nan LIN, Taoyuan County (TW); Chung Ping CHUANG, Taoyuan County (TW); Che Liang TSAI, Taichung City (TW); Kun Cheng HSIEH, Taipei City (TW)

(72) Inventors: Chun Nan LIN, Taoyuan County (TW); Chung Ping CHUANG, Taoyuan County (TW); Che Liang TSAI, Taichung City (TW); Kun Cheng HSIEH, Taipei City (TW)

(73) Assignee: CRYSTALVUE MEDICAL CORPORATION, Taoyuan County (TW)

(21) Appl. No.: 13/584,469

(22) Filed: Aug. 13, 2012

(30) Foreign Application Priority Data
Oct. 28, 2011 (TW) 100139263

(51) Int. Cl. A61B 3/15; A61B 3/12

(52) U.S. Cl. 351/208

(57) ABSTRACT

A simplified and cost-effective three-axis positioning device and method for ophthalmic examination instrument is disclosed. The three-axis positioning device includes an illuminating optical path for projecting light to illuminate an examinee's fundus; an imaging optical path including an objective lens for receiving the examinee's fundus image and light reflected from the examinee's cornea and eye-lens; a software-based alignment module for determining intensity and position of the reflected light on the fundus image to generate auxiliary positioning information; and an image displaying unit for showing the fundus image, the reflected light, and the auxiliary positioning information. From the intensity and position of the reflected light, x-, y- and z-axis relative positions between the examinee's pupil and the objective lens are obtained. An examiner adjusts the relative positions in three axes until they fall within an allowable deviation range, and a clear fundus image can be obtained.

### FIELD OF THE INVENTION

The present invention relates to a device and method for accurate positioning of an examinee's pupil relative to an ophthalmic examination instrument, and more particularly to a three-axis positioning device and method for ophthalmic examination instrument that uses a software-based alignment module to replace the conventional positioning optical path system.

### BACKGROUND OF THE INVENTION

When using a conventional fundus camera, which is a type of ophthalmic examination instrument, it is necessary to accurately adjust the relative positions between the examinee's pupil and the camera in three axes, that is, x-axis, y-axis and z-axis. Without such accurate three-axis positioning for pupil, most part of the illuminating light could not enter the pupil to reach the fundus while a part of the illuminating light would be reflected from the eye onto a fundus image formed on the CCD (Charge Coupled Device) in the fundus camera.

Please refer to FIG. 1. In the event of a poor three-axis positioning, there would be very intense reflected illuminating light shown around the fundus image formed on the CCD. On the other hand, in the event of a good three-axis positioning, the whole fundus image formed on the CCD would have uniform color as shown in FIG. 2, and no reflected illuminating light is shown around the fundus image.

The conventional fundus camera uses an additional positioning optical path system to achieve the three-axis positioning of the examinee's pupil relative to the fundus camera. Please refer to FIG. 3. The conventional fundus camera is aligned with the pupil 11 of an examinee's eye 10 for shooting a picture of the examinee's fundus 12. For this purpose, the conventional fundus camera mainly includes a light source projecting system 20, an optical camera system 21, an image displaying and monitoring system 22, and a positioning optical path system 23.

The light source projecting system 20 includes a photographing light source 200, a monitoring light source 201, condenser lens 202, 203, a beam splitter 204, an annular slit plate 205, and a relay lens 206. The optical camera system 21 includes an objective lens 210, a perforated lens 211, a magnifier 212, a film 213, a beam-bending lens 214, a field lens 215, a reflecting mirror 216, a relay lens 217, and a converter tube 218. The image displaying and monitoring system 22 includes a monitor 220 connected to the converter tube 218. The positioning optical path system 23 includes a semi-lens 230, a relay lens 231, a reflecting mirror 232, a light guide 233, a light source 234, and a focusing lens 235.

In the conventional fundus camera, the provision of the positioning optical path system 23 between the light source projecting system 20 and the optical camera system 21 would increase the complexity in the overall optical path design of the ophthalmic examination instrument. The conventional fundus camera must have additional space for the positioning optical path system 23, which also results in additional designing cost therefor.

In view that the use of the positioning optical path system in the conventional fundus camera for three-axis positioning would increase the overall cost of the whole ophthalmic examination instrument, it is desirable to improve the conventional ophthalmic examination that uses a positioning optical path system.

### SUMMARY OF THE INVENTION

A primary object of the present invention is to provide an improved three-axis positioning device for ophthalmic examination instrument, which includes a software-based alignment module for computing position and intensity of light reflected from an examinee's cornea and eye-lens onto an examinee's fundus image and determining whether the examinee's pupil is accurately aligned with an objective lens of the ophthalmic examination instrument in x-, y- and z-axis, so that the conventional hardware-based positioning optical path system can be omitted from the ophthalmic examination instrument to simplify the structure and reduce the manufacturing cost thereof.

To achieve the above and other objects, the three-axis positioning device for ophthalmic examination instrument according to an embodiment of the present invention includes an illuminating optical path, an imaging optical path, an image displaying unit, and a completely software-based alignment module. The illuminating optical path projects illuminating light to illuminate an examinee's fundus. The imaging optical path includes an objective lens for receiving light reflected from the examinee's cornea and eye-lens as well as the examinee's fundus image. The image displaying unit is connected to the imaging optical path for displaying the fundus image as well as the light reflected from the cornea and eye-lens. The alignment module is connected to the image displaying unit for determining intensity and position of the light reflected from the cornea and eye-lens shown on the fundus image to obtain relative positions between the examinee's pupil and the objective lens in three axes, and generates auxiliary x-axis, y-axis and z-axis positioning information, which is displayed on the image displaying unit to help an examiner to make adjustment and accordingly align the examinee's pupil with the objective lens.

In a preferred embodiment, the x-axis and y-axis positioning information displayed on the image displaying unit includes a set of coordinate axes, an allowable deviation range and a position indicating point; and the z-axis positioning information is shown on the image displaying unit by changing a diameter of the position indicating point.

In another preferred embodiment, the z-axis positioning information displayed on the image display unit includes indicating words and/or an indicating light.

According to the present invention, the light reflected from the examinee's cornea and eye-lens is shown on the fundus image as a first annular zone having a thickness within a predetermined thickness range when the examinee's pupil is correctly aligned with the objective lens; or as a substantially crescent zone or a second annular zone having a thickness non-matching the predetermined thickness range when the examinee's pupil is excessively deviated from the objective lens.

Another object of the present invention is to provide a three-axis positioning method for ophthalmic examination instrument, in which intensity, position and area size of light reflected from an examinee's cornea and eye-lens onto the examinee's fundus image are computed to generate auxiliary x-, y- and z-axis positioning information, based on which an examiner adjusts the relative positions between the examinee's pupil and an objective lens of the ophthalmic examination instrument in three axes, so as to accurately align the examinee's pupil with the objective lens.

To achieve the above and other objects, the three-axis positioning method for ophthalmic examination instrument according to an embodiment of the present invention includes the following steps: projecting illuminating light onto an examinee's fundus to illuminate the same; receiving light reflected from the examinee's cornea and eye-lens as well as an image of the examinee's fundus; detecting x-axis, y-axis and z-axis relative positions between the examinee's pupil and the ophthalmic examination instrument according to intensity and position of the light reflected from the examinee's cornea and eye-lens shown on the fundus image; and adjusting the relative positions between the examinee's pupil and the ophthalmic examination instrument according to the detected x-, y- and z-axis relative positions until a position deviation between the pupil and the ophthalmic examination instrument falls within an allowable deviation range.

In a preferred embodiment, the step of detecting the x-axis and y-axis relative positions between the examinee's pupil and the ophthalmic examination instrument further includes the following steps: dividing the fundus image into a first, a second, a third and a fourth quadrant using a horizontal line and a vertical line passing through a central point of the fundus image; using a preset first radius and a preset second radius to define a first area, a second area, a third area and a fourth area in the first, the second, the third and the fourth quadrant, respectively; obtaining the x-axis relative position by deducting a total brightness of the two areas at the left side of the vertical line from a total brightness of the two areas at the right side of the vertical line, and obtaining the y-axis relative position by deducting a total brightness of the two areas below the horizontal line from a total brightness of the two areas above the horizontal line; and displaying the x-axis and y-axis relative positions, and determining whether these two relative positions fall within the allowable deviation range.

In an operable embodiment, the step of determining whether the x-axis and y-axis relative positions fall within the allowable deviation range further includes the following steps: showing the x-axis and y-axis relative positions as a set of coordinate axes; setting an accuracy circle on the set of coordinate axes corresponding to the allowable deviation range; marking a position indicating point on the set of coordinate axes according to the x-axis and y-axis relative positions; and verifying whether the position indicating point is located within the accuracy circle on the set of coordinate axes.

In another preferred embodiment, the step of detecting the z-axis relative position between the examinee's pupil and the ophthalmic examination instrument further includes the following steps: using a threshold value of the illuminating light as a basis to set an allowable thickness range for the light circle formed around the fundus image by the light reflected from the examinee's cornea and eye-lens; shooting an actual fundus image, and capturing an actual thickness of the reflected light circle around the actual fundus image; and determining whether the actual thickness of the reflected light circle falls within the allowable thickness range and displaying the determined result.

In an operable embodiment, the step of determining whether the z-axis relative position falls within the allowable deviation range further includes the following steps: forming the z-axis relative position into an indicating area by way of datamation; setting indicating words and/or an indicating light on the indicating area corresponding to the allowable deviation range; and directly showing in the indicating area whether the z-axis relative position falls within the allowable deviation range.

According to the present invention, the indicating words are changeable in contents to indicate a distance state of the z-axis relative position between the examinee's pupil and the ophthalmic examination instrument, and the indicating light is changeable in its color to indicate the distance state of the z-axis relative position between the examinee's pupil and the ophthalmic examination instrument. Alternatively, the indicating light is changeable in its diameter to indicate a distance state of the z-axis relative position between the examinee's pupil and the ophthalmic examination instrument.

The present invention is characterized by a software-based alignment module added to the three-axis positioning device for computing the intensity, position and area size of light reflected from the examinee's cornea and eye-lens at the time of forming the examinee's fundus image, so as to provide the examiner with auxiliary positioning information for determining whether the examinee's eye has been accurately positioned in x-, y- and z-axis to align with the objective lens of the three-axis positioning device. The examiner can adjust the relative positions between the examinee's pupil and the examination instrument according to the displayed auxiliary positioning information to achieve accurate pupil alignment, while the conventional complicated hardware-based positioning optical path system can be saved from the ophthalmic examination instrument to simplify the design and reduce the manufacturing cost thereof.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 shows an examinee's fundus image formed on a conventional ophthalmic examination instrument when the examinee's pupil is in an excessively deviated position;

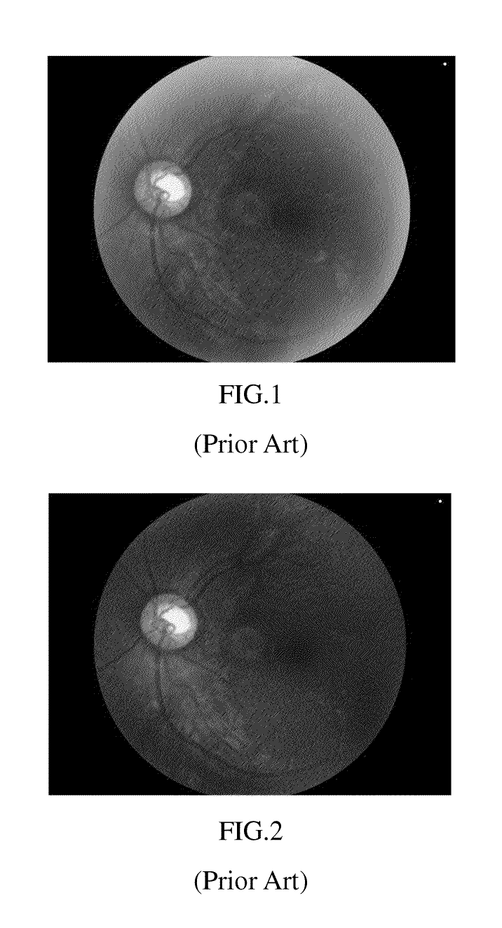

FIG. 2 shows an examinee's fundus image formed on a conventional ophthalmic examination instrument when the examinee's pupil is in a correctly aligned position;

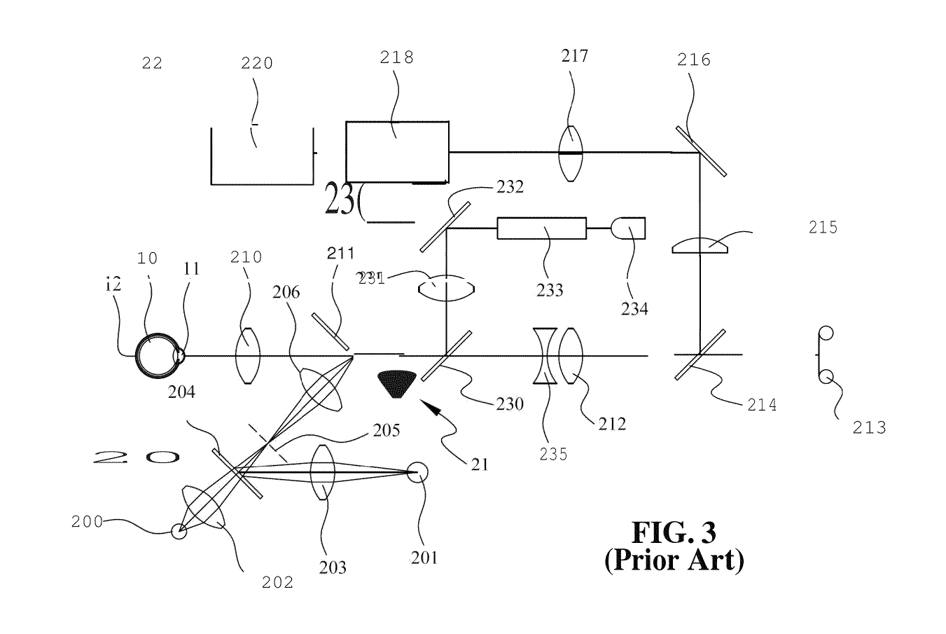

FIG. 3 is a conceptual view showing an overall optical path structure for a conventional ophthalmic examination instrument;

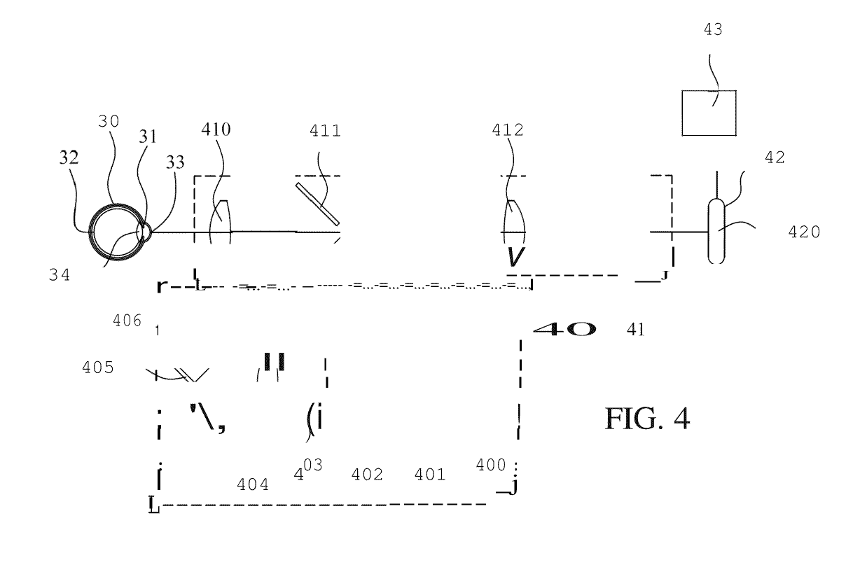

FIG. 4 is a conceptual view showing the structure of a three-axis positioning device for ophthalmic examination instrument according to an embodiment of the present invention;

FIGS. 5 and 5A to 5F are an examinee's fundus images taken with the examinee's pupil at different positions relative to the three-axis positioning device;

FIG. 6 is an examinee's fundus image taken with the present invention, on which auxiliary three-axis positioning information is shown;

FIG. 7 is a flowchart showing the steps included in a three-axis positioning method for ophthalmic examination instrument according to an embodiment of the present invention;

FIG. 8 is a flowchart showing the steps included in the method of the present invention for detecting x-axis and y-axis relative positions between an examinee's eye and the ophthalmic examination instrument;

FIG. 9 schematically shows how an alignment module included in the present invention computes the intensity and position of light reflected from the examinee's eye onto the examinee's fundus image;

FIG. 10 shows a set of coordinate axes used in the present invention to show auxiliary x-axis and y-axis positioning information for aligning the examinee's eye with the ophthalmic examination instrument;

FIG. 11 is a flowchart showing the steps included in the method of the present invention for detecting the z-axis relative position between the examinee's eye and the ophthalmic examination instrument;

FIGS. 12A to 12C show indicating words and an indicating light are used in the present invention to show the auxiliary z-axis positioning information for aligning the examinee's eye with the ophthalmic examination instrument; and

FIGS. 13A to 13B show the use of changes in a diameter of the indicating light adopted in the present invention to show the auxiliary z-axis positioning information for aligning the examinee's eye with the ophthalmic examination instrument.

### DETAILED DESCRIPTION OF THE PREFERRED EMBODIMENTS

The present invention will now be described with some preferred embodiments thereof and with reference to the accompanying drawings.

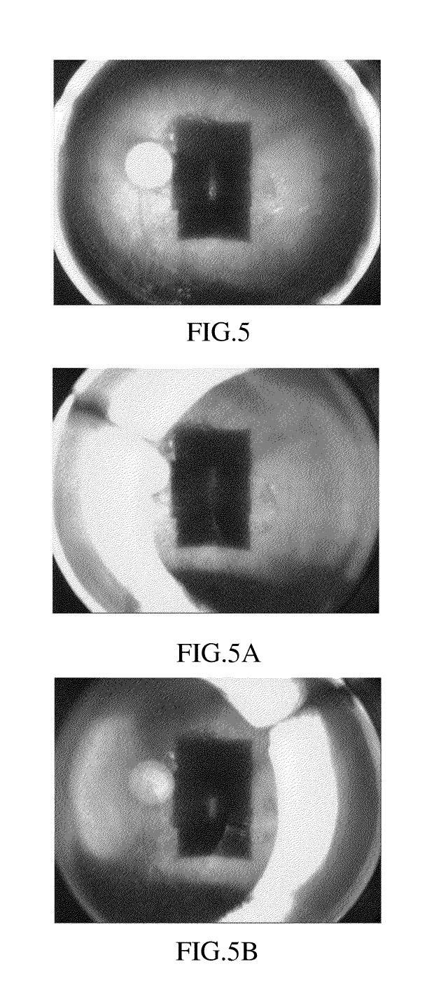

Please refer to FIG. 4 that is a conceptual view showing the structure of a three-axis positioning device for ophthalmic examination instrument according to an embodiment of the present invention. For the purpose of conciseness, the present invention is also briefly referred to as the three-axis positioning device herein. The three-axis positioning device of the present invention is configured for taking a picture of an examinee's eye fundus 32 via the pupil 31 of the examinee's eye 30. However, the present invention is characterized in that it determines the relative position between the examinee's pupil 31 and the ophthalmic examination instrument by light reflected from the examinee's cornea 33 and eye-lens 34. As shown in FIG. 4, the three-axis positioning device of the present invention includes an illuminating optical path 40, an imaging optical path 41, an image displaying unit 42, and a completely software-based alignment module 43.

In a preferred embodiment, the illuminating optical path 40 can include an illuminating light source 400, a condenser lens 401, a relay lens 402, an eye-lens diaphragm 403, a relay lens 404, a reflecting mirror 405, and a relay lens 406.

In the illuminating optical path 40, illuminating light emitted from the illuminating light source 400 is first projected onto the condenser lens 401 and the relay lens 402 to pass through the eye-lens diaphragm 403, and is then projected onto the reflecting mirror 405 via another relay lens 404. The reflecting mirror 405 changes the direction of the illuminating light, so that the illuminating light passes through the further relay lens 406 and enters into the imaging optical path 41 to illuminate the fundus 32 of the examinee's eye 30.

The imaging optical path 41 can includes an objective lens 410, a perforated mirror 411, and a focusing lens 412. The objective lens 410 in the imaging optical path 41 receives an image of the examinee's fundus 32 and light reflected from the examinee's cornea 33 and eye-lens 34. Under the effect of the perforated mirror 411 and of the focusing lens 412, the fundus image and the light reflected from the cornea 33 and the eye-lens 34 are formed on the image displaying unit 42.

The image displaying unit 42 includes a monitor 420 aligned with the focusing lens 412. The monitor 412 can be a CCD (Charge Coupled Device) image sensor.

The alignment module 43 is a program module connected to the image displaying unit 42 for determining intensity and position of the light reflected from the cornea 33 and the eye-lens 34 onto the fundus image, so as to obtain relative positions between the examinee's pupil 31 and the objective lens 410 in three axes and to generate auxiliary x-axis positioning information, y-axis positioning information and z-axis positioning information to the image displaying unit 42 for displaying. In this manner, the monitor 420 can simultaneously display the fundus image, the light reflected from the cornea 33 and the eye-lens 34, and the auxiliary positioning information for pupil alignment.

With the alignment module 43 provided in the present invention, an ophthalmic examination instrument without any positioning optical path system may directly utilize the information shown on the image displaying unit 42 to adjust the relative positions between the pupil 31 and the objective lens 410. That is, the present invention allows an examiner to intuitively adjust the relative positions of the examinee's pupil 31 to align the same with the objective lens 410.

From the above description, it can be understood the present invention is characterized by adding a completely software-based alignment module 43 to the imaging optical path 41, so that the alignment module 43 directly captures the positional deviation of the pupil 31 relative to the objective lens 410 and directly shows such positional deviation on the image displaying unit 42. It is to be understood the above-described hardware members for the illuminating optical path 40, the imaging optical path 41 and the image displaying unit 42 are only illustrative and not intended to limit the present invention in any way. That is, the hardware members in the optical paths according to the present invention can be adjusted according to actual need in use.

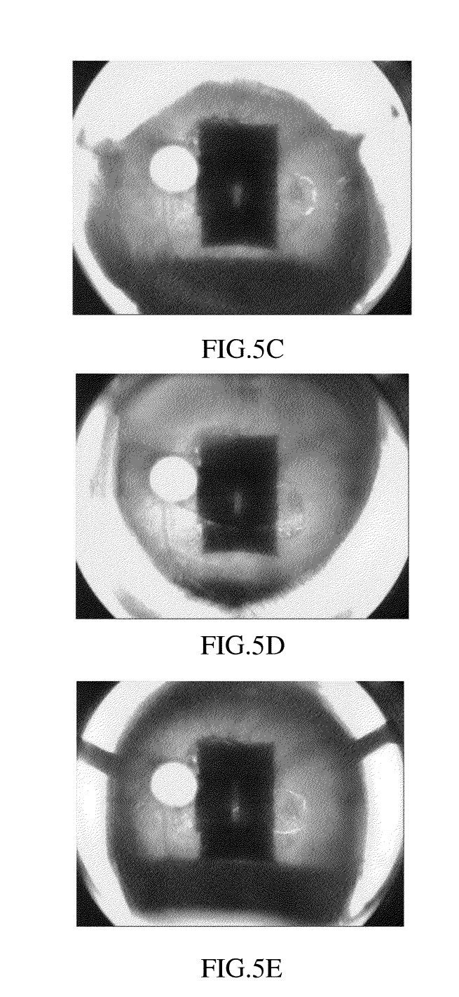

Please refer to FIG. 5. In an accurately shot fundus image, there is a light circle of a predetermined thickness located around the fundus image, and the light circle is formed by the light reflected from the examinee's cornea 33 and eye-lens 34. Please further refer to FIGS. 5A to 5F. In the event the examinee's pupil 31 is laterally deviated (i.e. leftward or rightward deviated) from the objective lens 410, the light reflected from the examinee's cornea 33 and eye-lens 34 will form an intense reflected-light zone on a contrary side, i.e. the right side or the left side, of the fundus image, respectively. For example, in FIG. 5A, since the pupil 31 is rightward deviated from a center of the objective lens 410, the intense reflected-light zone is formed closer to the left side of the fundus image. On the other hand, in FIG. 5B, since the pupil 31 is leftward deviated from a center of the objective lens 410, the intense reflected-light zone is formed closer to the right side of the fundus image.

In the event the examinee's pupil 31 is vertically deviated (i.e. upward or downward deviated) from the objective lens 410, the light reflected from the examinee's cornea 33 and eye-lens 34 will form an intense reflected-light zone on a contrary side, i.e. the lower side or the upper side, of the fundus image, respectively, as shown in FIGS. 5C and 5D.

Further, it is possible the examinee's pupil 31 is axially unsuitably located in front of the objective lens 410, i.e. the examinee's pupil 31 is axially too distant from or too close to the objective lens 410. In the event the examinee's pupil 31 is axially too distant from the objective lens 410, the light reflected from the examinee's cornea 33 and eye-lens 34 will form a reflected-light zone around the fundus image and the reflected-light zone has a thickness larger than the above-mentioned predetermined thickness for the reflected-light circle, as shown in FIG. 5E. On the other hand, in the event the examinee's pupil 31 is axially too close to the objective lens 410, the light reflected from the examinee's cornea 33 and eye-lens 34 will form a reflected-light zone around the fundus image and the reflected-light zone has a thickness smaller than the above-mentioned predetermined thickness, as shown in FIG. 5F.

In brief, the reflected-light zone is shown as a first annular zone having a thickness within a predetermined thickness range when the examinee's pupil is correctly aligned with the objective lens; or as a substantially crescent zone or as a second annular zone having a thickness different from the predetermined thickness range when the examinee's pupil is excessively deviated from the objective lens.

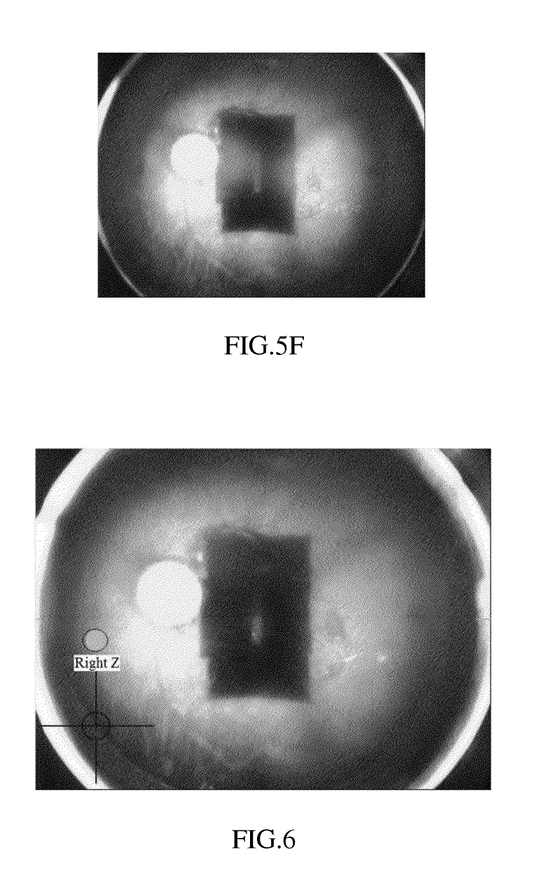

Please refer to FIG. 6. In a preferred embodiment of the present invention, the fundus image shown on the image displaying unit 42 includes x-axis, y-axis and z-axis positioning information displayed near a lower left corner of the fundus image. The x-axis and y-axis positioning information includes a set of coordinate axes, an allowable deviation range and a position indicating point; and the z-axis positioning information includes indicating words and/or an indicating light.

In another preferred embodiment, the z-axis positioning information is shown on the image displaying unit 42 by changing the size of the above-mentioned position indicating point.

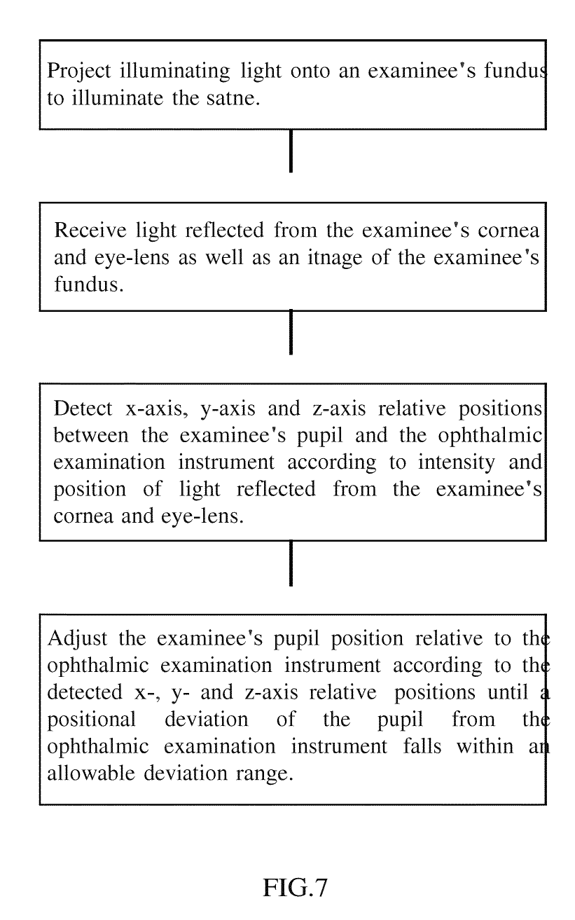

The present invention also provides a three-axis positioning method for ophthalmic examination instrument, steps of which are described below with reference to the flowchart of FIG. 7:
(i) Project illuminating light onto an examinee's fundus to illuminate the same;
(ii) Receive light reflected from the examinee's cornea and eye-lens as well as an image of the examinee's fundus;
(iii) Detect x-axis, y-axis and z-axis relative positions between the examinee's pupil and the ophthalmic examination instrument according to intensity and position of light reflected from the examinee's cornea and eye-lens; and
(iv) Adjust the examinee's pupil position relative to the ophthalmic examination instrument according to the detected x-, y- and z-axis relative positions until a positional deviation of the pupil from the ophthalmic examination instrument falls within an allowable deviation range.

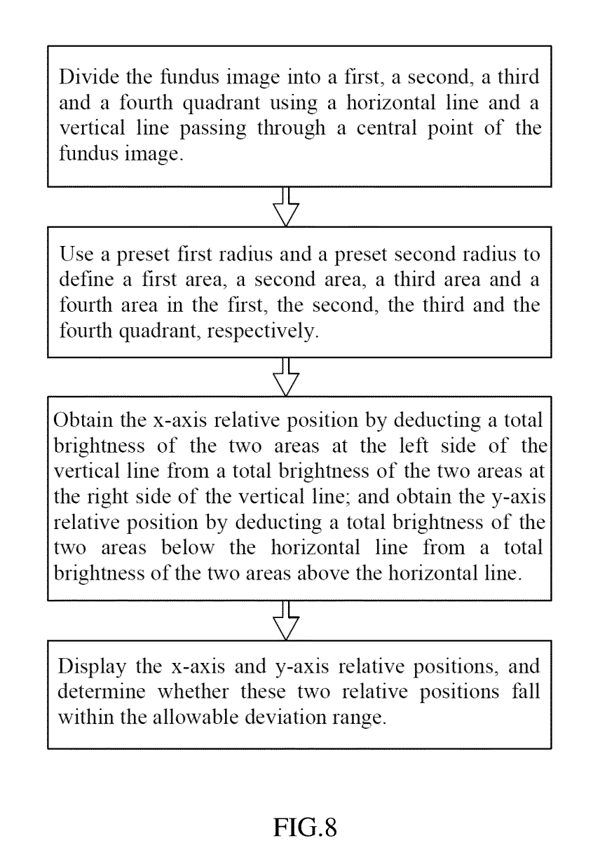

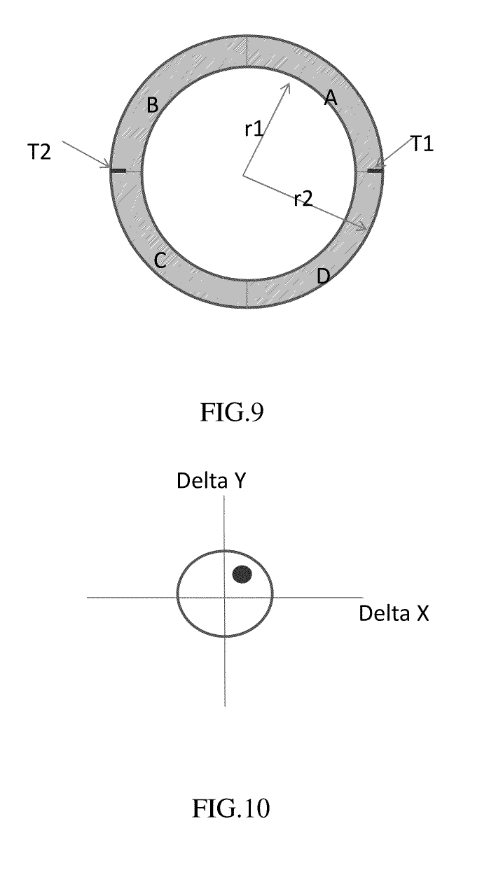

Please refer to FIGS. 8 and 9. In a preferred embodiment of the three-axis positioning method for ophthalmic examination instrument according to the present invention, the following steps are further included for detecting the x-axis and y-axis relative positions between the examinee's pupil and the ophthalmic examination instrument:
(iii-1) Divide the fundus image into a first, a second, a third and a fourth quadrant using a horizontal line and a vertical line passing through a central point of the fundus image;
(iii-2) Use a preset first radius and a preset second radius to define a first area, a second area, a third area and a fourth area in the first, the second, the third and the fourth quadrant, respectively. In an embodiment illustrated in FIG. 9, radius r1, radius r2, a horizontal line and a vertical line are used to define four areas A, B, C and D in the first to the fourth quadrants, respectively;
(iii-3) Obtain the x-axis relative position by deducting a total brightness of the two areas at the left side of the vertical line from a total brightness of the two areas at the right side of the vertical line; and obtain the y-axis relative position by deducting a total brightness of the two areas below the horizontal line from a total brightness of the two areas above the horizontal line. Where, a total brightness of the area A is defined as La, a total brightness of the area B is defined as Lb, a total brightness of the area C is defined as Lc, and a total brightness of the area D is defined as Ld; and the x-axis relative position=[(La+Ld)−(Lb+Lc)]/Sum, and the y-axis relative position=[(La+Lb)−(Lc+Ld)]/Sum, where Sum=La+Ld+Lb+Lc; and
(iii-4) Display the x-axis and y-axis relative positions, and determine whether these two relative positions fall within the allowable deviation range.

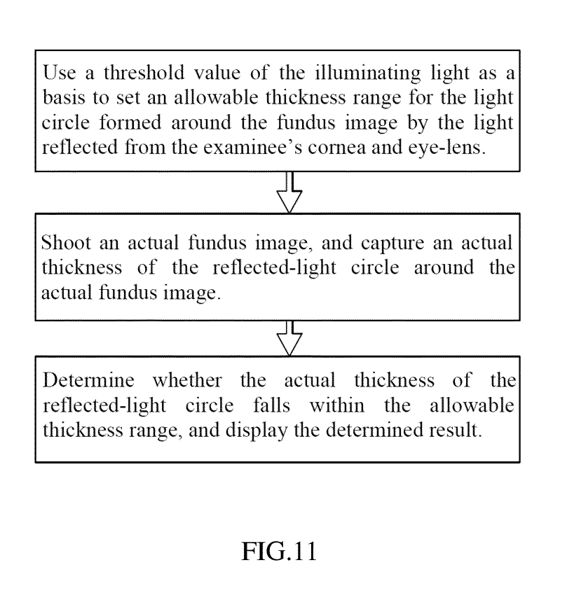

Please refer to FIG. 10. According to an operable embodiment of the present invention, the following steps are included to determine whether the x-axis and y-axis relative positions fall within the allowable deviation range: show the x-axis and y-axis relative positions as a set of coordinate axes; set an accuracy circle on the set of coordinate axes corresponding to the allowable deviation range; mark a position indicating point on the set of coordinate axes according to the x-axis and y-axis relative positions; and verify whether the position indicating point is located within the accuracy circle on the set of coordinate axes.

When the image display unit 42 shows on its operating interface a red point that is located within the accuracy circle representing the allowable deviation range, as shown in FIG. 10, the x-axis and y-axis positioning of the examinee's pupil relative to the ophthalmic examination instrument is completed.

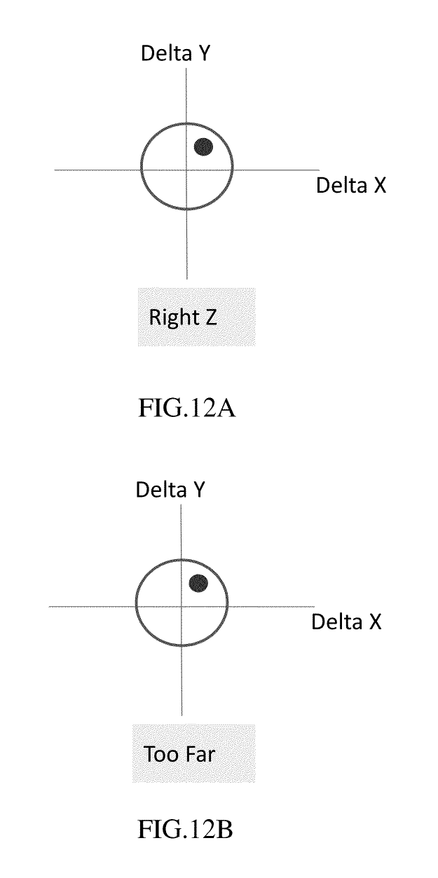

Please refer to FIG. 11. In a preferred embodiment of the three-axis positioning method for ophthalmic examination instrument according to the present invention, the following steps are further included for detecting the z-axis relative position between the examinee's pupil and the ophthalmic examination instrument:
(iii-5) Use a threshold value of the illuminating light as a basis to set an allowable thickness range for the light circle formed around the fundus image by the light reflected from the examinee's cornea and eye-lens. In an operable embodiment of the present invention, the threshold value for the illuminating light is set to a grayscale value of 150, so that thickness T1, T2 larger than the threshold value can be obtained at the horizontal position, as shown in FIG. 9;
(iii-6) Shoot an actual fundus image, and capture an actual thickness of the reflected-light circle around the actual fundus image; and
(iii-7) Determine whether the actual thickness of the reflected-light circle falls within the allowable thickness range, and display the determined result.

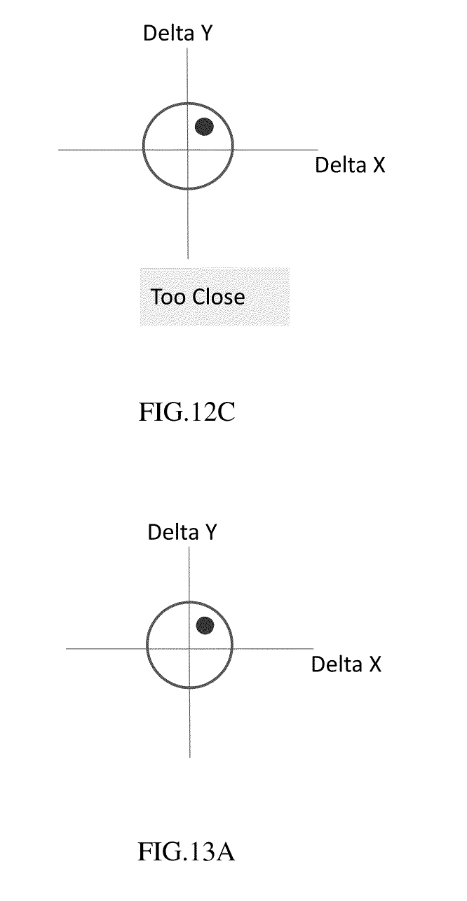

Please refer to FIGS. 12A to 12C. According to an operable embodiment of the present invention, the following steps are included to determine whether the z-axis relative position falls within the allowable deviation range: form the z-axis relative position into an indicating area by way of datamation; set indicating words and/or an indicating light on the indicating area corresponding to the allowable deviation range; and directly show in the indicating area whether the z-axis relative position falls within the allowable deviation range.

As shown at a lower portion in each of FIGS. 12A to 12C, the indicating words can have different contents to indicate a distance state of the z-axis relative position. In an operable embodiment, the indicating words can be one of “Right Z”, “Too Far” and “Too Close” to remind the examiner of the actual z-axis relative position. Further, the above-mentioned indicating light can show different colors to indicate different distance states of the z-axis relative positions.

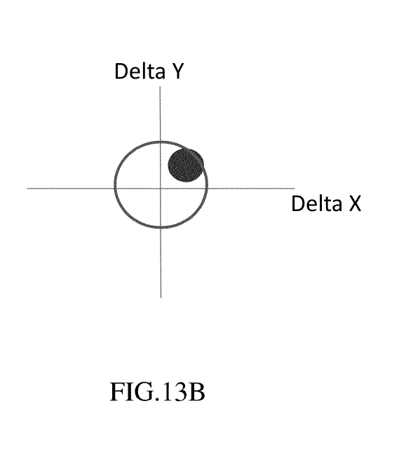

Please refer to FIGS. 13A and 13B. In another preferred embodiment, the indicating light can show changes in its diameter to directly indicate whether the z-axis relative position falls within the allowable deviation range.

In conclusion, in the present invention, a completely software-based alignment module is added to the ophthalmic examination instrument for computing the intensity, position and area size of the light reflected from the examinee's cornea and eye-lens at the time of forming the examinee's fundus image, so as to provide the examiner with auxiliary three-axis positioning information for determining whether the examinee's eye has been accurately positioned in x-, y- and z-axis to align with the ophthalmic examination instrument. The examiner can adjust the relative positions between the examinee's pupil and the examination instrument according to the displayed auxiliary three-axis positioning information to achieve accurate pupil alignment, while the conventional hardware-based complicated positioning optical path system can be saved from the ophthalmic examination instrument to simplify the design and reduce the manufacturing cost thereof.

The present invention has been described with some preferred embodiments thereof and it is understood that many changes and modifications in the described embodiments can be carried out without departing from the scope and the spirit of the invention that is intended to be limited only by the appended claims.

What is claimed is:

1. A three-axis positioning device for ophthalmic examination instrument, comprising:
an illuminating optical path for projecting illuminating light to illuminate an examinee's fundus;
an imaging optical path including an objective lens for receiving light reflected from the examinee's cornea and eye-lens as well as the examinee's fundus image;
an image displaying unit connected to the imaging optical path for displaying the fundus image as well as the light reflected from the cornea and eye-lens; and
an alignment module being electrically connected to the image displaying unit for determining intensity and position of the light reflected from the cornea and eye-lens onto the fundus image to obtain relative positions between the examinee's pupil and the objective lens;
wherein the alignment module generates auxiliary x-axis, y-axis and z-axis positioning information, which is displayed on the image displaying unit to help an examiner make adjustment and accordingly align the examinee's pupil with the objective lens.

2. The three-axis positioning device for ophthalmic examination instrument as claimed in claim 1, wherein the x-axis and y-axis positioning information displayed on the image displaying unit includes a set of coordinate axes, an allowable deviation range and a position indicating point.

3. The three-axis positioning device for ophthalmic examination instrument as claimed in claim 2, wherein the z-axis positioning information is displayed on the image displaying unit by changing a diameter of the position indicating point.

4. The three-axis positioning device for ophthalmic examination instrument as claimed in claim 1, wherein the z-axis positioning information displayed on the image displaying unit includes indicating words and/or an indicating light.

5. The three-axis positioning device for ophthalmic examination instrument as claimed in claim 1, wherein the light reflected from the examinee's cornea and eye-lens is shown on the fundus image as a first annular zone having a thickness within a predetermined thickness range when the examinee's pupil is correctly aligned with the objective lens; or as a substantially crescent zone or a second annular zone having a thickness different from the predetermined thickness range when the examinee's pupil is excessively deviated from the objective lens.

6. A three-axis positioning method for ophthalmic examination instrument, comprising the following steps:
projecting illuminating light onto an examinee's fundus to illuminate the same;
receiving light reflected from the examinee's cornea and eye-lens as well as an image of the examinee's fundus;
detecting x-axis, y-axis and z-axis relative positions between the examinee's pupil and the ophthalmic examination instrument according to intensity and position of the light reflected from the examinee's cornea and eye-lens shown on the fundus image; and
adjusting the examinee's pupil position relative to the ophthalmic examination instrument according to the detected x-, y- and z-axis relative positions until a positional deviation of the pupil from the ophthalmic examination instrument falls within an allowable deviation range.

7. The three-axis positioning method for ophthalmic examination instrument as claimed in claim 6, wherein the step of detecting the x-axis and y-axis relative positions between the examinee's pupil and the ophthalmic examination instrument further includes the following steps:
dividing the fundus image into a first, a second, a third and a fourth quadrant using a horizontal line and a vertical line passing through a central point of the fundus image;
using a preset first radius and a preset second radius to define a first area, a second area, a third area and a fourth area in the first, the second, the third and the fourth quadrant, respectively;
obtaining the x-axis relative position by deducting a total brightness of the two areas at the left side of the vertical line from a total brightness of the two areas at the right side of the vertical line; and obtaining the y-axis relative position by deducting a total brightness of the two areas below the horizontal line from a total brightness of the two areas above the horizontal line; and
displaying the x-axis and y-axis relative positions, and determining whether these two relative positions fall within the allowable deviation range.

8. The three-axis positioning method for ophthalmic examination instrument as claimed in claim 7, wherein the step of determining whether the x-axis and y-axis relative positions fall within the allowable deviation range further includes the following steps:
showing the x-axis and y-axis relative positions as a set of coordinate axes;
setting an accuracy circle on the set of coordinate axes corresponding to the allowable deviation range;
marking a position indicating point on the set of coordinate axes according to the x-axis and y-axis relative positions; and
verifying whether the position indicating point is located within the accuracy circle on the set of coordinate axes.

9. The three-axis positioning method for ophthalmic examination instrument as claimed in claim 6, wherein the step of detecting the z-axis relative position between the examinee's pupil and the ophthalmic examination instrument further includes the following steps:
using a threshold value of the illuminating light as a basis to set an allowable thickness range for the light circle formed around the fundus image by the light reflected from the examinee's cornea and eye-lens;
shooting an actual fundus image, and capturing an actual thickness of the reflected-light circle around the actual fundus image; and
determining whether the actual thickness of the reflected-light circle falls within the allowable thickness range and displaying the determined result.

10. The three-axis positioning method for ophthalmic examination instrument as claimed in claim 9, wherein the step of determining whether the z-axis relative position falls within the allowable deviation range further includes the following steps:
forming the z-axis relative position into an indicating area by way of datamation;
setting indicating words and/or an indicating light on the indicating area corresponding to the allowable deviation range; and
directly showing in the indicating area whether the z-axis relative position falls within the allowable deviation range.

11. The three-axis positioning method for ophthalmic examination instrument as claimed in claim 10, wherein the indicating words are changeable in contents to indicate a distance state of the z-axis relative position between the examinee's pupil and the ophthalmic examination instrument.

12. The three-axis positioning method for ophthalmic examination instrument as claimed in claim 10, wherein the indicating light is changeable in its color to indicate a distance state of the z-axis relative position between the examinee's pupil and the ophthalmic examination instrument.

13. The three-axis positioning method for ophthalmic examination instrument as claimed in claim 10, wherein the indicating light is changeable in its diameter to indicate a distance state of the z-axis relative position between the examinee's pupil and the ophthalmic examination instrument.
